(19) United States
(12) Patent Application Publication
(10) Pub. No.: US 2026/0014293 A1
(43) Pub. Date: Jan. 15, 2026

(54) HEMOSTATIC AGENT IN PASTE FORM, USE THEREOF, AND METHOD FOR PRODUCING A LOCAL ACTIVE AGENT RELEASE SYSTEM

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/259,900

(22) Filed: Jul. 3, 2025

(30) Foreign Application Priority Data
Jul. 9, 2024 (EP) 24187301.7

(51) Int. Cl. A61L 26/00

(52) U.S. Cl. CPC A61L 26/0052; A61L 26/0066; A61L 2300/406; A61L 2400/04; A61L 2400/06; A61L 2430/02

(57) ABSTRACT

A hemostatic agent in paste form is proposed. The hemostatic agent comprises at least one particulate sugar alcohol; at least one saturated triglyceride having a melting point greater than or equal to 40° C.; and, at least one saturated triglyceride having a melting point of less than 0° C. Furthermore, the uses of the hemostatic agent in paste form and a method for its preparation are described.

### CROSS-REFERENCE TO RELATED APPLICATION

This application claims priority pursuant to 35 U.S.C. 119(a) to European Patent Office Application No. 24187301.7, filed Jul. 9, 2024, which application is incorporated herein by reference in its entirety.

### BACKGROUND

The present invention relates to a hemostatic agent in paste form which can be used for the mechanical sealing of bleeding bone tissue. A further object of the present invention is an active agent release system which is based on the hemostatic agent in paste form. The invention further relates to the uses of the hemostatic agent according to the invention and of the active agent release system according to the invention, as well as to methods for producing the hemostatic agent according to the invention and the active agent release system according to the invention.

### SUMMARY OF INVENTION

During operations, according to the anatomical conditions, bleeding is stopped (hemostasis) using different methods, such as electrocoagulation (cauterization) of the blood vessels. In several operations in the skull area and especially on the sternum, due to the anatomical situation, bone wax is used to seal the capillaries and thus stop the bleeding when severe bleeding occurs there. For this purpose, the surgeon presses the plastically deformable bone wax directly onto or into the bleeding bone areas. This leads to congestion of the blood flow, which causes thrombocyte aggregation; and the supplying vessels are ultimately blocked by platelet aggregation and fibrin.

Bone wax has been known since at least the 19th century, and generally contains bleached beeswax and a plasticizer. Almond oil, petroleum jelly, palmitic acid, isopropyl ester and isopropyl myristic acid are among the plasticizers used. Bone waxes based on beeswax are considered non-biodegradable in the human organism. Due to the chemical composition of the beeswax used, bone wax is not broken down by human enzymes. As a result, the bone wax remains in or on the bone tissue after hemostasis and constitutes a barrier to the growth of new bone tissue.

The hemostatic effect of bone waxes is due to their good adhesion to moist and fatty bone tissue and their high toughness. The bone waxes currently available on the market have a very good hemostatic effect.

However, there are often undesirable side effects and consequences in the human organism over longer periods of time (SE Katz, J. Rotmann: Adverse effects of bone wax in surgery of the orbit. Ophthal Plast. Reconstr. 1996, 12 (2) 121-126.; M. Lavigne et al.: Bone-wax granuloma of old femoral neck osteoplasty. Can. J. Surg. 2008, 51 (3) E58-60.; RT Allison: Foreign body reactions and an associated histological artifact due to bone wax. Br. J. Biomed. Sci. 1994, 51 (1) 14-17.; O. Eser et al.: Bone wax as a cause of foreign body reaction alter lumbar disc surgery: A case report. Adv. Ther. 2007, 24 (3) 594-7).

Alternatives to conventionally composed bone wax are known.

EP 0 109 310 A discloses a waxy mass based on calcium fatty acid salts and oligomers of hydroxycarboxylic acids.

The documents U.S. Pat. No. 4,595,713, DE 322 95 40, DE 382 52 11 and EP 1 142 597 disclose waxy compositions which contain oligoesters of hydroxycarboxylic acids, such as lactic acid and 6-hydroxycarboxylic acid. It has been shown that when these waxy compositions are used, acidic degradation products are formed during hydrolytic degradation, which can affect the bone tissue as a result of a local pH reduction.

An alternative are compositions based on polyethers (US 2009/286886 and US 2011/002974). Polypropylene glycol/ethylene glycol copolymers, for example, can be used as polyethers. These compositions become kneadable and spreadable with hand warmth. However, the good solubility of these polyethers in aqueous media is a disadvantage. This means that the adhesion of these compositions may be difficult in the case of heavily bleeding bone tissue due to the dissolution of the waxy composition. This may result in subsequent bleeding, which in turn may cause the seal to dissociate or dissolve quickly. The advantage of these mixtures, however, is that they have no barrier function for bone healing and are completely excreted via the kidneys (A. Suwan et al.: Controversial role of two different local hemostatic agents on bone healing. J. am Sci. 2010, 6 (12) 15-163).

Patents DE 10 2011 016277 and DE 10 2011 122 752 describe a hemostatic agent in paste form. The hemostatic agent is composed of (a) at least one saturated glycerol-1,2,3-trifatty acid ester having a melting temperature of more than 37° C., (b) at least one filler present at least partially in particulate form having a melting temperature of more than 37° C. and (c) at least one compound having a melting temperature of not more than 37° C. which has a solubility of less than 50 grams per liter of water at a temperature of 25° C. Polymers of at least one alkylene oxide, copolymers of at least one alkylene oxide and calcium compounds are proposed as fillers. Calcium compounds described include calcium carbonate, dolomite, a-tricalcium carbonate, β-tricalcium carbonate, hydroxyapatite, carbonate apatite, octacalcium phosphate, amorphized calcium phosphate, calcium sulfate dihydrate and calcium sulfate hemihydrate. Polyethylene glycols and polypropylene glycol/polyethylene glycol copolymer (poloxamers) are preferred as polymers. Liquid fatty acid esters constituted the third component. It was shown in our own research on in vitro cytotoxicity according to ISO1993-5 updated that a hemostatic agent according to the patent specifications DE 10 2011 016 277 and DE 10 2011 122 752, which is composed of a solid triglyceride, a liquid triglyceride and the fillers calcium carbonate and calcium sulfate, is not cytotoxic according to ISO10993-5. In contrast, hemostatics with a similar composition, but with polypropylene glycol/polyethylene glycol copolymer (poloxamer) as filler instead of the inorganic calcium salts, are clearly cytotoxic. The calcium compounds are also disadvantageous due to their abrasive properties.

There is therefore a need for a plastically deformable, biodegradable hemostatic agent that generally does not have the disadvantages described above.

In particular, there is a need for hemostatics in paste-form that consist entirely of non-abrasive organic, biocompatible substances. Abrasive components are particularly critical if the hemostatic agent is used in the vicinity of joint endoprostheses, because they can cause abrasive damage to the sliding surfaces if the abrasive particles get between the articulating surfaces of the joint endoprostheses.

The object of the invention is to develop a hemostatic agent in paste form that preferably consists of biocompatible and resorbable materials and does not present in vitro cytotoxicity according to ISO10993-5. In this context, it is also particularly preferred if the hemostatic agent in paste form according to the invention does not have amphiphilic properties. Amphiphilic substances penetrate both hydro-and lipophilic components of a cell and thus weaken the cell membrane. This means that they can increase cytotoxicity. It is also intended that it does not contain any abrasive, inorganic calcium salts. In addition, the hemostatic agent to be developed should not contain any components that can be used by microorganisms as an energy source. Furthermore, the hemostatic agent should not release any acidic or basic components in large quantities, to avoid damaging the bone tissue due to a non-physiological pH value. In addition, the material should be biodegradable or able to be excreted renally so that no permanent barrier effect from the material can hinder the healing method of the bone tissue. The hemostatic agent should be easily malleable by hand. In addition, the paste-form material should not stick to rubber gloves during kneading and application. It is intended to adhere to moist bone tissue and also to the surfaces of metallic implants. Furthermore, it is intended that the hemostatic agent can be mixed with any powdered pharmaceutical active agents without significantly impairing the adhesive properties and the plastic deformability of the hemostatic agent. The viscosity of this hemostatic agent should be high enough so that the paste-form material can withstand the bleeding pressure. Furthermore, the hemostatic agent should have sufficient cohesion so that it does not fall apart or dissolve within a few minutes when it comes into contact with blood or other aqueous fluids.

The objects of the invention are firstly achieved by the hemostatic agent according to the invention.

The present invention initially relates to a hemostatic agent in paste form. The hemostatic agent is characterized by the fact that it contains
a) at least one particulate sugar alcohol,
b) at least one saturated triglyceride having a melting point greater than or equal to 40° C., and,
c) at least one saturated triglyceride having a melting point of less than 0° C.

The individual components of the hemostatic agent according to the invention are described below.

In a preferred embodiment, the hemostatic agent according to the invention comprises, in each case based on the total weight of the hemostatic agent,
a) 30-60 wt. %, preferably 40-60 wt. %, more preferably 50-60 wt. %, of at least one particulate sugar alcohol,
b) 20-35 wt. %, preferably 20-30 wt. %, more preferably 20-28 wt. %, of at least one saturated triglyceride having a melting point greater than or equal to 40° C., and,
c) 20-35 wt. %, preferably 20-30 wt. %, more preferably 20-28 wt. %, of at least one saturated triglyceride having a melting point of less than 0° C.

The hemostatic agent according to the invention has, for example, the following advantages.

The hemostatic agent in paste form according to the invention is preferably non-cytotoxic according to ISO10993-5, determined by the MTT test ((3-(4,5-dimethylthiazol-2-yl)-2,5-diphenyltetrazolium bromide) test) or by the XTT test (((sodium 3′-[1-(phenylaminecarbonyl)-3,4-tetrazolium]-bis-(4-methoxy-6-nitro))-benzenesulfonic acid hydrate) test).

The hemostatic agent in paste form according to the invention is characterized by its plastic deformability, biodegradability and resistance to a liquid, in particular aqueous, environment. It remains dimensional and volume-stable in the absence of external force, especially ex vivo. Notwithstanding this, the hemostatic agent is degradable as described herein.

The hemostatic agent in paste form according to the invention is suitable for the treatment of damaged bone tissue and can serve both for mechanical hemostasis and as a local active agent release center in paste form. The hemostatic agent according to the invention generally adheres equally to bone, glass and metal.

In addition, it is possible to add a solid or preferably particulate pharmaceutical active agent to the hemostatic agent according to the invention. In this embodiment, the hemostatic agent, when used to treat damage to bone tissue, can serve as a local active agent release center in paste form.

The hemostatic agent in paste form according to the invention is generally accessible by simply kneading its components on a laboratory or industrial scale.

### i) Hemostatic Agent in Paste Form

According to the invention, a hemostatic agent is provided.

According to the invention, a hemostatic agent is understood to mean a composition which has hemostatic properties.

The invention is based on the surprising discovery that mixtures of components a), b) and c) as defined above form a hemostatic agent in paste form which can be used to seal bleeding bone tissue. What is particularly surprising is that the hemostatic agent according to the invention is present as a waxy, kneadable mass which generally adheres to both dry and moist surfaces. The hemostatic agent adheres particularly to metallic surfaces, glass and bone tissue. The toughness and mechanical stability of this mixture are surprisingly high enough to be used as an effective hemostatic agent for stopping bleeding and to withstand the bleeding pressures encountered in injuries. Although the mixture is generally biodegradable, it surprisingly exhibits such great mechanical stability that it does not decompose upon contact with water or aqueous solutions, such as blood.

In the context of the present invention, substances that can be broken down by the human organism and/or excreted renally are referred to as biodegradable.

The hemostatic agent according to the invention comprises at least one sugar alcohol.

Sugar alcohols, such as mannitol, generally do not affect the insulin levels of patients treated with the hemostatic agent according to the invention. The sugar alcohols of the hemostatic agent according to the invention also have a surprising influence on its paste-form consistency. Without being bound to any theory, they reduce the crystal growth of the other possible components during the manufacturing process of the hemostatic agent in paste form according to the invention by wetting the crystallite surfaces formed and thus making them inaccessible to further crystal-forming molecules. As a result, the crystallites remain small and the hemostatic agent in paste form does not form chunks but rather remains in the paste form according to the invention, which has a uniform consistency. According to the invention, it is assumed that sugar alcohols are excreted undegraded from the human body renally with the blood without causing any damage. In addition, they have a particularly positive influence on the nature of the hemostatic agent in paste form and are thus a surprisingly useful component of the hemostatic agent in paste form according to the invention.

The hemostatic agent according to the invention comprises triglycerides as a further component. In the context of the present invention, triglycerides are understood to be organic compounds which are cleaved by lipases to glycerol and fatty acids. As a natural component of the human organism, glycerin is generally broken down into carbon dioxide and water via pyruvate and the citric acid cycle. Fatty acids are also natural components of the human organism and, in the case of fatty acids with an even number of C atoms, are generally completely broken down into carbon dioxide and water via β-oxidation.

Further definitions of the individual components of the hemostatic agent according to the invention can be found below.

The hemostatic agent according to the invention is plastically deformable. In this context, plastic deformability is understood to mean the ability of the hemostatic agent to irreversibly deform under the application of a force and to maintain this shape after the application of the force.

A further advantage of the hemostatic agent in paste form according to the invention is that it generally has no cytotoxic properties. In the context of the present invention, this is preferably achieved by using only those substances or mixtures of substances in the hemostatic agent according to the invention which have an in vitro cytotoxicity according to ISO10993-5 with a viability of greater than 70% at 100% v/v in the MTT test ((3-(4,5-dimethylthiazol-2-yl)-2,5-diphenyltetrazolium bromide) test) or in the XTT test (((sodium 3′-[1-(phenylaminecarbonyl)-3,4-tetrazolium]-bis-(4-methoxy-6-nitro))-benzenesulfonic acid hydrate) test). This means that in the MTT or XTT test according to ISO10993-5, the hemostatic agent in paste form according to the invention has a viability of preferably greater than 70.0%, 70.5%, 71.0%, 71.5%, 72.0%, 72.5%, 73.0%, 73.5%, 74.0%, 74.5%, 75.0%, 75.5%, 76.0%, 76.5%, 77.0%, 77.5%, 78.0%, 78.5%, 79.0%, 79.5%, 80.0%, 80.5%, 81.0%, 81.5%, 82.0%, 82.5%, 83.0%, 83.5%, 84.0%, 84.5%, 85.0%, 85.5%, 86.0%. 86.5%, 87.0%, 87.5%, 88.0%, 88.5%, 89.0%, 89.5%, 90.0%, 90.5%, 91.0%, 91.5%, 92.0%, 92.5%, 93.0%, 93.5%, 94.0%, 94.5% or 95.0% at 100% v/v. It is preferred that at least a viability between 70.0% and 80.0% is achieved at 100% v/v.

### ii) Composition of the Hemostatic Agent in Paste Form

The hemostatic agent in paste form comprises at least one particulate sugar alcohol.

In the context of the present invention, a sugar alcohol is understood to mean a non-cyclic polyol which has a hydroxy group bonded to each of its carbon atoms. Sugar alcohols suitable according to the invention are generally obtained as reduction products of carbohydrates (sugars), i.e. by reduction (hydrogenation) of the keto group or the aldehyde group of sugars. As a result, sugar alcohols are generally much more oxidation-stable and storage-stable than the parent sugars. Furthermore, they generally have little or no effect on the blood sugar concentration in the human organism and are not or only partially usable as an energy source by many microorganisms.

Particulate sugar alcohols in the context of the invention are preferably alditols and ketols, with particular preference being given to D-mannitol (CAS 69-65-8), D,L-mannitol (CAS 87-78-5), D-sorbitol (CAS 50-70-4), isomalt (6-OaD-glucopyranosyl-D-glucitol) (CAS 534-73-6), 1-OaD-glucopyranosil-D-mannitol (CAS 20942-99-8), erythritol (CAS 149-32-6) and xylitol (CAS 87-99-0). More-preferred sugar alcohols are erythritol, xylitol, D-mannitol and D,L-mannitol. D-mannitol and D,L-mannitol are particularly preferably used in the context of the present invention.

Experiments according to the invention with different sugar alcohols show that D-mannitol is particularly suitable in the context of the present invention with regard to haptic properties and with regard to the in vitro cytotoxicity according to ISO10993-5. D-mannitol also has no effect on blood sugar levels and is hemocompatible.

Sugar alcohols generally do not contain any acidic or basic groups. Therefore, they have virtually no effect on the pH of aqueous solutions or water when dissolved in them or when they come into contact with the aqueous solutions or water. Experiments show that the pH value of distilled water in which the hemostatic agent in paste forms according to the invention is in the range of pH 6.0-6.8.

For the purposes of the invention, the sugar alcohols as component a) are preferably present in the form of particulate sugar alcohols. This means that the particulate sugar alcohols are preferably in the form of solid particles, which are characterized by having a particle diameter of preferably less than 100 μm. The particles of the at least one particulate sugar alcohol are therefore smaller than preferably 100 μm, 99 vm, 98 μm, 97 μm, 96 μm, 95 μm, 94 μm, 93 μm, 92 μm, 91 μm, 90 μm, 89 μm, 88 μm, 87 μm, 86 μm, 85 μm, 84 μm, 83 μm, 82 μm, 81 μm, 80 μm, 79 μm, 78 μm, 77 μm, 76 μm, 75 μm, 74 μm, 73 μm, 72 μm, 71 μm, 70 μm, 69 μm, 68 μm, 67 μm, 66 μm, 65 μm, 64 μm, i.e. 63 μm, 62 μm, 61 μm, 60 μm, 59 μm, 58 μm, 57 μm, 56 μm, 55 μm, 54 μm, 53 μm, 52 μm, 51 μm or 50 μm. In the present invention, the particle diameter is determined by sieve fractionation.

The smaller the particle size of the sugar alcohol, the smoother the paste that can be produced from it.

A further component of the hemostatic agent in paste form in the context of the present invention are saturated triglycerides with a melting point greater than or equal to 40° C. as component b). Such saturated triglycerides are generally solid at room temperature. Furthermore, their melting point is above the normal human body temperature of about 36 to 38° C., making them a possible component of the hemostatic agent according to the invention, which is also solid under in vivo conditions.

Particularly suitable saturated triglycerides with a melting point greater than or equal to 40° C. in the context of the present invention are glycerol tristearate (CAS 55-43-1), glycerol tripalmitate (CAS 555-44-2), glycerol trilaurate (CAS 555-44-2) and mixed triglycerides formed from stearate and/or palmitate and/or laurate and glycerol. However, other possible types of saturated fatty acid esters are also conceivable, which have a melting point greater than or equal to 40° C. and enable the previously described, preferred properties of the hemostatic agent according to the invention with regard to surface adhesion, deformability, toughness, biodegradability, etc. These saturated fatty acid esters with a melting point greater than or equal to 40° C. are preferred for the adhesion of the hemostatic agent to bone tissue and to metallic surfaces. Furthermore, these saturated fatty acid esters generally have high storage stability and high resistance to gamma irradiation because they do not contain double bonds or other oxidation-sensitive structures. Therefore, the particulate hemostatic agent according to the invention is particularly suitable for treating injuries or surgically induced impairments of the bones. Any X-rays following treatment with the particulate hemostatic agent of the invention generally do not damage the area treated with the particulate hemostatic agent. In particular, the hemostatic agent according to the invention can also be used to treat bone damage in patients who subsequently have to undergo radiotherapy. The irradiation preferably cannot affect the particulate hemostatic agent according to the invention.

A further component of the hemostatic agent in paste form in the context of the present invention are saturated triglycerides with a melting point below 0° C. as component c). Such saturated triglycerides are liquid at room temperature, i.e. between 20° C. and 30° C.

Particularly suitable saturated triglycerides with a melting point below 0° C. in the context of the present invention are glycerol trioctanoate (CAS 538-23-8) and glycerol tridecanoate (CAS 612-71-6), with mixtures of glycerol fatty acids being particularly preferred and the glycerol fatty acid mixtures Miglyol 812 N (CAS 73398-61-5), Miglyol 810 N (CAS 73398-61-5) and Miglyol 829 N (Cas 91744-56-8) being very particularly preferred. These liquid fatty acid esters also do not contain double bonds or other oxidation-sensitive structures and are therefore generally storage-stable and largely resistant to gamma radiation. The use of triglycerides of which the fatty acid moieties have an even number of C atoms is particularly advantageous. If there is an even number of C atoms, fatty acids are completely metabolized into carbon dioxide and water in the human organism.

According to the invention, it is preferred if the different components a) (particulate sugar alcohols), b) (saturated glycerol fatty acid esters with a melting point greater than or equal to 40° C.) and c) (saturated glycerol fatty acid esters with a melting point below 0° C.) are each present in a certain proportion in the hemostatic agent according to the invention. According to the invention, it is possible to vary the respective proportions of the components in the hemostatic agent in paste-form according to the selected substances and the intended application of the hemostatic agent in paste-form.

For the purposes of the present invention, it has been shown that component a) preferably makes up 30% to 60 wt. % of the total mass of the hemostatic agent in paste form. This means that the particulate sugar alcohol, regardless of the other components, makes up a proportion of preferably at least 30 wt. %, generally at least 31 wt. %, generally at least 32 wt. %, generally at least 33 wt. %, generally at least 34 wt. %, generally at least 35 wt. %, generally at least 36 wt. %, generally at least 37 wt. %, generally at least 38 wt. %, generally at least 39 wt. %, generally at least 40 wt. %, generally at least 41 wt. %, generally at least 42 wt. %, generally at least 43 wt. %, generally at least 44 wt. %, generally at least 45 wt. %, generally at least 46 wt. %, generally at least 47 wt. %, generally at least 48 wt. %, generally at least 49 wt. %, generally at least 50 wt. %, in each case based on the total mass of the hemostatic agent according to the invention. Furthermore, this means that the particulate sugar alcohol, independently of the other components, makes up a proportion of preferably at most 60 wt. %, generally at most 59 wt. %, generally at most 58 wt. %, generally at most 57 wt. %, generally at most 56 wt. %, generally at most 55 wt. %, generally at most 54 wt. %, generally at most 53 wt. %, generally at most 52 wt. %, generally at most 51 wt. %, generally at most 50 wt. %, in each case based on the total mass of the hemostatic agent according to the invention. It is particularly preferred if component a), i.e. the particulate sugar alcohol, is present in a range from 40 wt. % to 60 wt. %, even more preferably from 50 wt. % to 60 wt. %, in each case based on the total mass of the hemostatic agent according to the invention.

For the purposes of the present invention, it has been shown that components b) and c) preferably each make up 20 wt. % to 35 wt. % of the total mass of the hemostatic agent in paste form.

This means that the saturated triglyceride with a melting point greater than or equal to 40° C., independently of the other components, makes up a proportion of preferably at least 20 wt. %, generally at least 21 wt. %, generally at least 22 wt. %, generally at least 23 wt. %, generally at least 24 wt. %, generally at least 25 wt. %, generally at least 26 wt. %, generally at least 27 wt. %, generally at least 28 wt. %, generally at least 29 wt. %, generally at least 30 wt. %, in each case based on the total mass of the hemostatic agent according to the invention. Furthermore, this means that the saturated triglyceride with a melting point greater than or equal to 40° C., independently of the other components, makes up a proportion of preferably at most 35 wt. %, generally at most 34 wt. %, generally at most 33 wt. %, generally at most 32 wt. %, generally at most 31 wt. %, generally at most 30 wt. %, in each case based on the total mass of the hemostatic agent according to the invention.

This also means that the saturated triglyceride with a melting point of less than 0° C., independently of the other components, makes up a proportion of preferably at least 20 wt. %, generally at least 21 wt. %, generally at least 22 wt. %, generally at least 23 wt. %, generally at least 24 wt. %, generally at least 25 wt. %, generally at least 26 wt. %, generally at least 27 wt. %, generally at least 28 wt. %, generally at least 29 wt. %, generally at least 30 wt. %, in each case based on the total mass of the hemostatic agent according to the invention. Furthermore, this means that the saturated triglyceride with a melting point of less than 0° C., independently of the other components, makes up a proportion of preferably at most 35 wt. %, generally at most 34 wt. %, generally at most 33 wt. %, generally at most 32 wt. %, generally at most 31 wt. %, generally at most 30 wt. %, in each case based on the total mass of the hemostatic agent according to the invention.

The hemostatic agent in paste form according to the invention comprises in particular, based on the total weight of the hemostatic agent in paste form,
a) 30-60 wt. %, preferably 40-60 wt. %, more preferably 50-60 wt. %, of at least one particulate sugar alcohol selected from the group consisting of D-mannitol, D,L-mannitol, xylitol and erythritol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate,
c) 20-35 wt. %, preferably 20-30 wt. %, more preferably 20-28 wt. %, of at least one saturated triglyceride having a melting point of less than 0° C., selected from the group consisting of glycerol fatty acid mixtures.

The hemostatic agent in paste form according to the invention comprises in particular, based on the total weight of the hemostatic agent in paste form,
d) 30-60 wt. %, preferably 40-60 wt. %, more preferably 50-60 wt. %, of at least one particulate sugar alcohol selected from the group consisting of D-mannitol, D,L-mannitol, xylitol and erythritol;
e) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate, and,
f) 20-35 wt. %, preferably 20-30 wt. %, more preferably 20-28 wt. %, of at least one saturated triglyceride having a melting point of less than 0° C., selected from the group consisting of the glycerol fatty acid mixtures Miglyol 812 N, Miglyol 810 N and Miglyol 829 N.

The hemostatic agent in paste form according to the invention comprises in particular, based on the total weight of the hemostatic agent in paste form,
a) 30-60 wt. %, preferably 40-60 wt. %, more preferably 50-60 wt. %, of at least one particulate sugar alcohol selected from the group consisting of D-mannitol and D,L-mannitol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate; and,
c) 20-35 wt. %, preferably 20-30 wt. %, more preferably 20-28 wt. %, of at least one saturated triglyceride having a melting point of less than 0° C., selected from the group consisting of glycerol fatty acid mixtures.

The hemostatic agent in paste form according to the invention comprises in particular, based on the total weight of the hemostatic agent in paste form,
a) 30-60 wt. %, preferably 40-60 wt. %, more preferably 50-60 wt. %, of at least one particulate sugar alcohol selected from the group consisting of D-mannitol and D,L-mannitol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate; and,
c) 20-35 wt. %, preferably 20-30 wt. %, more preferably 20-28 wt. %, of at least one saturated triglyceride having a melting point of less than 0° C., selected from the group consisting of the glycerol fatty acid mixtures Miglyol 812 N, Miglyol 810 N and Miglyol 829 N.

The hemostatic agent in paste form according to the invention is used in particular in vivo for the treatment of all conceivable types of external damage to bone structures.

For this purpose, it may be particularly advantageous in the context of the invention to add at least one solid, preferably a particulate, pharmaceutical active agent to the hemostatic agent in paste form, in addition to the previously mentioned components a), b) and c). The solid or particulate pharmaceutical active agent can be dispersed in the hemostatic agent in paste form without changing its chemical form. In this way, the active agent can be released locally, preferably over a longer period of time, and can exert all possible healing effects on the possibly damaged bone tissue in its surroundings (sustained release). If the hemostatic agent according to the invention comprises an active agent, an active agent release system according to the invention results. The active agent can either be added during the manufacture of the hemostatic agent or can be added by a healthcare professional immediately prior to administration to a patient.

Therefore, the present invention also relates to an active agent release system, comprising
a) at least one particulate sugar alcohol;
b) at least one saturated triglyceride having a melting point greater than or equal to 40° C., and,
c) at least one saturated triglyceride having a melting point of less than 0° C.; and,
d) a particulate pharmaceutical active agent.

In a preferred embodiment, the active agent release system according to the invention comprises, in each case based on the total weight of the active agent release system,
a) 30-60 wt. %, preferably 40-60 wt. %, more preferably 50-60 wt. %, of at least one particulate sugar alcohol;
b) 20-35 wt. %, preferably 20-30 wt. %, more preferably 20-28 wt. %, of at least one saturated triglyceride having a melting point greater than or equal to 40° C.;
c) 20-35 wt. %, preferably 20-30 wt. %, more preferably 20-28 wt. %, of at least one saturated triglyceride having a melting point of less than 0° C.; and,
d) 0.5-15 wt. %, preferably 1-12 wt. %, more preferably 2-10 wt. %, of a particulate pharmaceutical active agent.

In principle, any pharmaceutical active agent is suitable for this purpose. According to the invention, anti-infectives, blood coagulation activators, fibrinolysis inhibitors, immunomodulators, steroid hormones and growth factors are particularly preferred. Especially gentamicin (CAS 1403-66-3), vancomycin (CAS 1404-90-6), tobramycin (CAS 32986-56-4), clindamycin (Cas 18323-44-9), colistin (CAS 1066-17-7), meropenem (CAS 96036 Mar. 29), metronidazole (CAS 443-48-1), caspofungin (CAS 162808-62-0), fluconazole (Cas 86386-73-4), amphotericin B (CAS 1397-89-3), calcium gluconate (CAS 299-28-5), tranexamic acid (CAS 11197-18-8), 6-amino-caproic acid (CAS 60-32-2), p-aminomethylbenzoic acid (CAS 150-13-0), prednisolone (CAS 50-24-8), dexamethasone (CAS 50-02-2) and cyclosporin A (CAS 79217-60-0) are preferred.

Therefore, the present invention relates in particular to an active agent release system comprising, in each case based on the total weight of the active agent release system,
a) 30-60 wt. %, preferably 40-60 wt. %, more preferably 50-60 wt. %, of at least one particulate sugar alcohol;
b) 20-35 wt. %, preferably 20-30 wt. %, more preferably 20-28 wt. %, of at least one saturated triglyceride having a melting point greater than or equal to 40° C.;
c) 20-35 wt. %, preferably 20-30 wt. %, more preferably 20-28 wt. %, of at least one saturated triglyceride having a melting point of less than 0° C.; and,
d) 0.5-15 wt. %, preferably 1-12 wt. %, more preferably 2-10 wt. % of a particulate pharmaceutical active agent selected from the group consisting of anti-infectives, blood coagulation activators, fibrinolysis inhibitors, immunomodulators, steroid hormones and growth factors.

Furthermore, the present invention relates to an active agent release system comprising, in each case based on the total weight of the active agent release system,
a) 30-60 wt. %, preferably 40-60 wt. %, more preferably 50-60 wt. %, of at least one particulate sugar alcohol;
b) 20-35 wt. %, preferably 20-30 wt. %, more preferably 20-28 wt. %, of at least one saturated triglyceride having a melting point greater than or equal to 40° C.;
c) 20-35 wt. %, preferably 20-30 wt. %, more preferably 20-28 wt. %, of at least one saturated triglyceride having a melting point of less than 0° C.; and,
d) 0.5-15 wt. %, preferably 1-12 wt. %, more preferably 2-10 wt. % of a particulate pharmaceutical active agent selected from the group consisting of gentamicin, vancomycin, tobramycin, clindamycin, colistin, meropenem, metronidazole, caspofungin, fluconazole, amphotericin B, calcium gluconate, tranexamic acid, 6-aminocaproic acid, p-aminomethylbenzoic acid, prednisolone, dexamethasone, daptomycin and cyclosporin A.

The active agent release system according to the invention contains a particulate pharmaceutical active agent selected from the aforementioned active agents. Of these aforementioned active agents, anti-infectives are particularly preferred. Even more preferred are antibiotics, especially the antibiotics gentamicin, vancomycin, clindamycin and daptomycin.

Therefore, the present invention relates in particular to an active agent release system comprising, in each case based on the total weight of the active agent release system,
a) 30-60 wt. %, preferably 40-60 wt. %, more preferably 50-60 wt. %, of at least one particulate sugar alcohol;
b) 20-35 wt. %, preferably 20-30 wt. %, more preferably 20-28 wt. %, of at least one saturated triglyceride having a melting point greater than or equal to 40° C.;
c) 20-35 wt. %, preferably 20-30 wt. %, more preferably 20-28 wt. %, of at least one saturated triglyceride having a melting point of less than 0° C.; and,
d) 0.5-15 wt. %, preferably 1-12 wt. %, more preferably 2-10 wt. % of a particulate antibiotic.

Furthermore, the present invention relates in particular to an active agent release system comprising, in each case based on the total weight of the active agent release system,
a) 30-60 wt. %, preferably 40-60 wt. %, more preferably 50-60 wt. %, of at least one particulate sugar alcohol;
b) 20-35 wt. %, preferably 20-30 wt. %, more preferably 20-28 wt. %, of at least one saturated triglyceride having a melting point greater than or equal to 40° C.;
c) 20-35 wt. %, preferably 20-30 wt. %, more preferably 20-28 wt. %, of at least one saturated triglyceride having a melting point of less than 0° C.; and,
d) 0.5-15 wt. %, preferably 1-12 wt. %, more preferably 2-10 wt. % of a particulate pharmaceutical active agent selected from the group consisting of gentamicin, vancomycin, clindamycin and daptomycin.

The active agent release system in paste form according to the invention comprises in particular, based on the total weight of the active agent release system,
a) 30-60 wt. %, preferably 40-60 wt. %, more preferably 50-60 wt. %, of at least one particulatel sugar alcohol selected from the group consisting of D-mannitol, D,L-mannitol, xylitol and erythritol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate;
c) 20-35 wt. %, preferably 20-30 wt. %, more preferably 20-28 wt. %, of at least one saturated triglyceride having a melting point of less than 0° C., selected from the group consisting of glycerol fatty acid mixtures; and,
d) 0.5-15 wt. %, preferably 1-12 wt. %, more preferably 2-10 wt. % of a particulate pharmaceutical active agent selected from the group consisting of gentamicin, vancomycin, clindamycin and daptomycin.

The active agent release system in paste form according to the invention comprises in particular, based on the total weight of the active agent release system,
a) 30-60 wt. %, preferably 40-60 wt. %, more preferably 50-60 wt. %, of at least one particulate sugar alcohol selected from the group consisting of D-mannitol, D,L-mannitol, xylitol and erythritol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate,
c) 20-35 wt. %, preferably 20-30 wt. %, more preferably 20-28 wt. %, of at least one saturated triglyceride having a melting point of less than 0° C., selected from the group consisting of the glycerol fatty acid mixtures Miglyol 812 N, Miglyol 810 N and Miglyol 829 N; and,
d) 0.5-15 wt. %, preferably 1-12 wt. %, more preferably 2-10 wt. %, each based on the total mass of the active agent release system according to the invention, of a particulate pharmaceutical active agent selected from the group consisting of gentamicin, vancomycin, clindamycin and daptomycin.

The active agent release system in paste form according to the invention comprises in particular, based on the total weight of the active agent release system,
a) 30-60 wt. %, preferably 40-60 wt. %, more preferably 50-60 wt. %, of at least one particulate sugar alcohol selected from the group consisting of
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate;
c) 20-35 wt. %, preferably 20-30 wt. %, more preferably 20-28 wt. %, of at least one saturated triglyceride having a melting point of less than 0° C., selected from the group consisting of glycerol fatty acid mixtures; and,
d) 0.5-15 wt. %, preferably 1-12 wt. %, more preferably 2-10 wt. % of a particulate pharmaceutical active agent selected from the group consisting of gentamicin, vancomycin, clindamycin and daptomycin.

The active agent release system in paste form according to the invention comprises in particular, based on the total weight of the active agent release system,
a) 30-60 wt. %, preferably 40-60 wt. %, more preferably 50-60 wt. %, of at least one particulate sugar alcohol selected from the group consisting of D-mannitol and D,L-mannitol;
b) 20-35 wt. %, preferably 20-30 wt. %, more preferably 20-28 wt. %, of at least one saturated triglyceride having a melting point greater than or equal to 40° C., selected from the group consisting of glycerol tripalmitate, glycerol tristearate and glycerol tribehenate,
c) 20-35 wt. %, preferably 20-30 wt. %, more preferably 20-28 wt. %, of at least one saturated triglyceride having a melting point of less than 0° C., selected from the group consisting of the glycerol fatty acid mixtures Miglyol 812 N, Miglyol 810 N and Miglyol 829 N; and,
d) 0.5-15 wt. %, preferably 1-12 wt. %, more preferably 2-10 wt. %, each based on the total mass of the active agent release system according to the invention, of a particulate pharmaceutical active agent selected from the group consisting of gentamicin, vancomycin, clindamycin and daptomycin.

According to the invention, it is therefore preferably provided that 0.5-15.0 wt. % of at least one solid pharmaceutical active agent, preferably a particulate pharmaceutical active agent, is added to the hemostatic agent in paste form to form the active agent release system according to the invention. This means that the pharmaceutical active agent, if added to the hemostatic agent in paste form, preferably accounts for 0.5 wt. % to 15.0 wt. % of the total mass of the active agent release system. Accordingly, the proportion of the particulate pharmaceutical active agent in the total mass of the active agent release system according to the invention can be at least 0.5 wt. %, at least 1.0 wt. %, at least 1.5 wt. %, at least 2.0 wt. %, at least 2.5 wt. %. In addition, the proportion of the particulate pharmaceutical active agent in the total mass of the active agent release system according to the invention can amount to at most 15 wt. %, at most 14 wt. %, at most 13 wt. %, at most 12 wt. %, at most 11 wt. %, at most 10 wt. %.

The active agent release system according to the invention preferably comprises 1 wt. % to 12 wt. %, particularly preferably 2 wt. % to 10 wt. % of the particulate pharmaceutical active agent.

The particulate hemostatic agent according to the invention or the active agent release system according to the invention with the respective components described is preferably designed such that it remains dimensionally stable in distilled water at room temperature over a period of at least 7 days under laboratory conditions. This makes it capable of maintaining its desired shape in an environment where it is exposed to the influence of polar liquids. This is the case, for example, when the particulate hemostatic agent according to the invention or the active agent release system according to the invention is applied to an injured bone in order to stop any bleeding that may occur.

An advantage of the invention is that no inorganic calcium salts or magnesium salts need to be added to the claimed particulate hemostatic agent or active agent release system. Hemostatics known in the prior art often contain these inorganic salts and have the disadvantage that they typically have a Mohs hardness≥2. Due to their high Mohs hardness, they have an abrasive effect on the material surrounding them. Because preferably no inorganic salts such as calcium salts and magnesium salts have to be added to the particulate hemostatic agent according to the invention or the active agent release system according to the invention, the particulate hemostatic agent according to the invention or the active agent release system according to the invention has no abrasive properties and can therefore also be used in the field of joint endoprostheses without the sliding surfaces being affected by abrasion.

In one embodiment of the present invention, the hemostatic agent according to the invention and the active agent release system according to the invention do not contain any inorganic calcium salts and magnesium salts. However, in the context of the present invention, it is still possible in a further embodiment to add calcium or magnesium salts with a Mohs hardness≥2.0 to the particulate hemostatic agent according to the invention or to the active agent release system according to the invention. In this case, the mass fraction of calcium or magnesium salts with a Mohs hardness≥2.0, in each case based on the particulate hemostatic agent according to the invention or the active agent release system according to the invention, preferably does not exceed 10.0 wt. %. This means that preferably less than 10.0 wt. %, more preferably less than 9.5 wt. %, more preferably less than 9.0 wt. %, more preferably less than 8.5 wt. %, more preferably less than 8.0 wt. %, more preferably less than 7.5 wt. %, more preferably less than 7.0 wt. %, more preferably less than 6.5 wt. %, more preferably less than 6.0 wt. %, more preferably less than 5.5 wt. %, more preferably less than 5.0 wt. % of calcium or magnesium salts with a Mohs hardness≥2.0, in each case based on the total weight of the hemostatic agent according to the invention or active agent release system according to the invention, are added.

According to the above statements, calcium or magnesium salts which have a Mohs hardness of less than 2, i.e. a Mohs hardness of in particular less than 2.0, in particular less than 1.9, in particular less than 1.8, in particular less than 1.7, in particular less than 1.6, in particular less than 1.5, in particular less than 1.4, in particular less than 1.3, in particular less than 1.2, in particular less than 1.1, in particular less than 1.0, in particular less than 0.9, in particular less than 0.8, in particular less than 0.7, in particular less than 0.6, in particular less than 0.5, in particular less than 0.4, in particular less than 0.3, in particular less than 0.2, in particular less than 0.1, can be added to the particulate hemostatic agent according to the invention or to the active agent release system according to the invention.

In summary, the hemostatic agent according to the invention and the active agent release system according to the invention have the following advantages:

The hemostatic agent in paste form according to the invention comprises biodegradable substances which together can form a deformable paste which can be irreversibly brought into a desired shape under the application of force. The paste is suitable for use in vivo, and when degraded there, it usually does not form acidic or basic degradation products that could cause local damage.

All kinds of conceivable particulate pharmaceutical active agents, as described above, can also be added to the hemostatic agent in paste form. In this variant, the resulting active agent release system is particularly suitable for the treatment of any type of bone tissue damage.

The hemostatic agent in paste form or active agent release system generally remains dimensionally stable for a long period of time after application, even when exposed to liquid medium. Furthermore, it is preferable that no inorganic particles are added to it, so that it does not have any abrasive properties, for example towards metal objects.

All these properties allow the use of the hemostatic agent in paste form and active agent release system according to the invention for the treatment of all types of damage to bone tissue.

### iii) Uses of the Hemostatic Agent in Paste Form

The hemostatic agent in paste form and the active agent release system generally adhere to bone tissue and metal surfaces when pressure is applied. This makes it possible, for example, to use the hemostatic agent in paste form to treat damaged bone tissue, e.g. by stopping bleeding from a bone, such as after a fracture, an operation or even an amputation, with the hemostatic agent in paste form.

Since it usually also adheres to metal surfaces, the hemostatic agent in paste form is also particularly suitable for mechanical hemostasis and/or as a local active agent release system in paste form that is adhered to bone tissue surfaces and to surfaces of metallic implants by applying pressure. The local active agent release system in paste form, when applied to a treated site on a bone, is generally capable of releasing the dispersed particulate pharmaceutical active agent. The local active agent release system is preferably adhered to the surfaces of knee joint endoprostheses, hip joint endoprostheses, shoulder joint endoprostheses, intramedullary nails and osteosynthesis plates, preferably under pressure. A great advantage of the hemostatic agent according to the invention is that, as a local active agent release system, it usually remains stationary after adhering in vivo. This allows solid, preferably particulate, pharmaceutical active agents dispersed in the local active agent release system to be released specifically at the desired location.

Furthermore, use of the hemostatic agent in paste form for filling bone cavities is possible according to the invention, wherein the hemostatic agent is particularly preferably used for filling previously infected and debrided medullary cavities and screw holes in the bone tissue. In particular, when the hemostatic agent in paste form is applied to such areas as a local active agent center, it can release the particulate pharmaceutical active agent and then, for example, alleviate inflammatory symptoms.

The hemostatic agent in paste form according to the invention is used in particular to mechanically stop bleeding of the bone in vivo. If a particulate pharmaceutical active agent is added to the hemostatic agent in paste form according to the invention, it can also serve as a local active agent release system in paste form at the site treated with it. There it can then continuously release the active pharmaceutical agent dispersed in it over a certain period of time.

Therefore, the present invention further relates to a hemostatic agent in paste form as described above or to an active agent release system in paste-form as described above for use as a drug, in particular for use in the treatment of damaged bone tissue.

The hemostatic agent in paste-form or the active agent release system in paste form can be used for the treatment of bone cavities by filling, preferably by filling infected and debrided, medullary spaces and screw holes in the bone tissue.

For this purpose, the hemostatic agent in paste form or active agent release system in paste-form can be adhered to bone tissue surfaces and to surfaces of metallic implants by applying pressure, wherein the hemostatic agent in paste form or active agent release system in paste form is preferably adhered to the surfaces of knee joint endoprostheses, hip joint endoprostheses, shoulder joint endoprostheses, intramedullary nails and osteosynthesis plates.

The hemostatic agent in paste form can be administered to a patient manually or using a dispensing device. Due to the triglycerides contained, good lubricity and squeezability of the hemostatic agent can be achieved, for example in syringes and similar squeezing dosing systems.

The compositions described herein can also be used to coat medical implants, such as spacers, artificial joints and osteosynthesis plates. For this purpose, compositions according to the invention can be provided, for example, as an application pen or syringe system.

### iv) Method for Producing The Hemostatic Agent in Paste Form and the Active Agent Release System

According to the invention, the hemostatic agent in paste form and active agent release system is produced by mixing all components.

This means that at least one particulate sugar alcohol a), at least one saturated triglyceride with a melting point greater than or equal to 40° C. b), at least one saturated triglyceride with a melting point below 0° C. c) and, optionally, a particulate pharmaceutical active agent are placed in a suitable container, such as a beaker or an industrial mixing container, and mixed.

Preferably, 30 wt. % to 60 wt. % of the particulate sugar alcohol, 20 wt. % to 35 wt. % of the saturated triglyceride having a melting point greater than or equal to 40° C., 20 wt. % to 35 wt. % of the saturated triglyceride having a melting point below 0° C. and optionally 0.5 to 15 wt. % of a particulate pharmaceutical active agent are weighed in.

Preferred proportions of the individual components have already been disclosed above and apply to the method according to the invention mutatis mutandis.

It may be advantageous in the context of the invention if the weighed components a), b), c) and optionally d) are first heated and pre-stirred at a lower speed. The heating is generally carried out above the melting temperature of the saturated triglyceride with a melting point greater than or equal to 40° C. Preferably, it is not so high that the chemical nature of the components used could possibly change. For example, it is between 50° C. and 90° C., i.e. in particular at 50° C., 51° C., 52° C., 53° C., 54° C., 55° C., 56° C., 57° C., 58° C., 59° C. or 60° C., 61° C., 62° C., 63° C., 64° C., 65° C., 66° C., 67° C., 68° C., 69° C. or 70° C., 71°° C., 72° C., 73° C., 74° C., 75° C., 76° C., 77° C., 78° C., 79° C. or 80° C., 81° C., 82° C., 83° C., 84° C., 85° C., 86° C., 87° C., 88° C., 89° C. or 90° C.

The pre-stirred mixture is then usually cooled and mixed at least once using a suitable mixer or stirrer at high speed. Speeds between 1500 min⁻¹ and 3000 min⁻¹ can be contemplated—for example 1500 min⁻¹, 1600 min⁻¹, 1700 min⁻¹, 1800 min⁻¹, 1900 min⁻¹, 2000 min⁻¹, 2100 min⁻¹, 2200 min⁻¹, 2300 min⁻¹, 2400 min⁻¹, 2500 min⁻¹, 2600 min⁻¹, 2700 min⁻¹, 2800 min⁻¹, 2900 min⁻¹ or 3000 min⁻¹. In principle, any mixer with which high stirring rates can be achieved, as shown, is suitable. For the purposes of the present invention, the stirring step can be carried out once at an elevated speed, or repeated multiple times.

In the context of the present invention, it is possible to add a particulate pharmaceutical active agent to the hemostatic agent in paste form in order to use it later as a local active agent release system in paste form. In such a case, the particulate pharmaceutical active agent can be weighed at the beginning together with the other components a), b) and c).

The method according to the invention for producing the hemostatic agent in paste form, which is to be used as a local active agent release system in paste form, is characterized in particular in that at least one pharmaceutical active agent present in the solid state, preferably particulate, is mixed with the hemostatic agent in paste form by kneading.

### BRIEF DESCRIPTION OF THE SEVERAL VIEWS OF THE DRAWINGS

FIG. 1 is a hemostatic agent in paste form produced after mixing all components; malleability in the hand is indicated; and,

FIG. 2 is a hemostatic agent in paste form which has been pressed onto the metal surface of a spatula. It is evident that the hemostatic agent in paste form adheres to a metal surface.

### DETAILED DESCRIPTION

### Exemplary Embodiments

For the exemplary embodiments, pharmaceutical-quality Lytrol micro, Poloxamer 185, glycerol tripalmitate, glycerol tristearate, glycerol tribehenate, Miglyol 812 N and Miglyol 810 N were used.

The pastes were prepared in a simple manner: first, all components were weighed into a beaker. The mixtures were then heated to 90° C. for 1 hour while stirring occasionally. After cooling, the mixtures were transferred into plastic containers and mixed with a speed mixer at 2000 revolutions for 60 seconds each. The mixing method was then repeated twice. Colorless paste-form masses were formed.

TABLE 1
Active agent release systems according to the invention
using D-mannitol and glycerol tripalmitate

| | | Glycerol | Miglyol | |
|---|---|---|---|---|
| | D-mannitol | tripalmitate | 812N | |
| Examples | [g] | [g] | [g} | Assessment |
| 1 | 9.1 | 4.6 | 4.4 | Very soft, adheres very well to |
| | | | | metal, glass and bone |
| 2 | 9.1 | 4.6 | 4.0 | Soft, slightly firmer than paste of |
| | | | | example 1. Adheres very well to |
| | | | | metal, glass and bone |
| 3 | 9.1 | 4.1 | 4.0 | Soft, slightly firmer than the |
| | | | | pastes of examples 1 and 2, |
| | | | | adheres similarly well as the |
| | | | | pastes of examples 1 and 2 |

TABLE 2
Active agent release systems according to the invention using D-mannitol
and glycerol tristearate as well as glycerol tribehenate:

| | | | | | |
|---|---|---|---|---|---|
| | | Glycerol | Glycerol | Miglyol | Miglyol |
| | D-mannitol | tristearate | tribehenate | 812N | 812 |
| Examples | [g] | [g] | [g] | [g] | [g] |

| | | | | |
|---|---|---|---|---|
| 4 | 9.1 | 4.6 | — | 4.4 |
| 5 | 9.1 | — | 4.6 | 4.0 |
| 6 | 9.1 | 4.6 | — | 4.4 |
| 7 | 9.1 | — | 4.6 | 4.0 |

TABLE 3
Active agent release systems according to the invention
using xylitol and erythritol and glycerol tripalmitate

| | | | | Glycerol | |
|---|---|---|---|---|---|
| | Xylitol | Erythritol | Glycerol | tristearate | Miglyol |
| Examples | [g] | [g] | tripalmitate | [g] | 812N |
| 8 | 9.1 | — | 4.6 | | 4.4 |
| 9 | 9.1 | — | 4.6 | | 4.4 |
| 10 | — | 9.1 | — | 4, | 4.4 |
| 11 | — | 9.1 | — | 4.6 | 4.4 |

TABLE 4
Reference examples according to the teaching
of patent specification DE 10 2011 016 277 B

| | Lytrol micro | CSCA | Glycerol | Miglyol | |
|---|---|---|---|---|---|
| | (poloxamer) | mixture | tripalmitate | 812N | |
| Examples | [g] | [g] | [g] | [g} | Assessment |
| 12 | 11.3 | | 5.6 | 9.0 | Very soft, significantly |
| | | | | | softer than the paste of |
| | | | | | example 9, adheres |
| | | | | | very well to metal, |
| | | | | | glass and bone tissue |

The CSCA mixture consists of 80.0 wt. % calcium sulfate dihydrate and 20.0 wt. % calcium carbonate.

### Examination of the In Vitro Cytotoxicity According to ISO10993-5

Pastes of examples 1, 2, and 12 were tested for their in vitro cytotoxicity. Furthermore, poloxamer 185 was included in the tests.

The determination of in vitro cytotoxicity according to ISO10993-5 was carried out using the MTT test by Eurofins BioPharma Product Testing Munich GmbH. The pastes were eluted in cell culture medium for 72 hours at 37° C. The eluates were then incubated undiluted and in three dilutions with L929 cells for 24 hours at 37° C. The vitality of the cells was then determined photometrically using tetrazolium chloride. The tetrazolium chloride is reduced by vital cells to the red-violet dye 1,3,5-triphenylformazan. In the paste of example 12, the similarly structured dye triphenyltetrazlium chloride (XTT test) was used instead of tetrazolium chloride, which is also reduced to a red-violet dye by vital cells. This dye is also used in ISO10993-5 for testing the in vitro cytotoxicity described.

TABLE 5
Results for in vitro cytotoxicity for selected examples

| | Viability [%] | | | |
|---|---|---|---|---|
| | Test, extract dilution | | | |
| Examples | 100% v/v | 69.6% v/v | 44.4% v/v | 29.6% v/v |
| 1 | 73 | 74 | 79 | 82 |
| 2 | 72 | 79 | 81 | 82 |
| 12 | 44 | 49 | 65 | 77 |
| Poloxamer 185 | 0 | 0 | 0 | 1 |

According to ISO10993-5, samples are considered non-cytotoxic if they have a viability of greater than 70% in the MTT test or the XTT test at 100% v/v. The pastes according to the invention of examples 1 and 2 were evaluated as non-cytotoxic according to the MTT test with 73% and 72% viability. The paste of example 12, which contains Poloxamer 185, was clearly cytotoxic with a viability of less than 70%.

The in vitro cytotoxicity of the pastes of examples 1 and 2 was then additionally tested with the agar diffusion test according to ISO109992-5 using L929 cells. Both pastes showed no cytotoxic effect.

### Assessment of the Admixability of Antibiotics

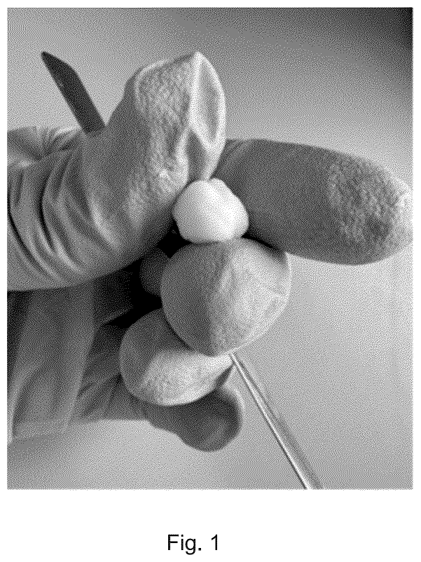

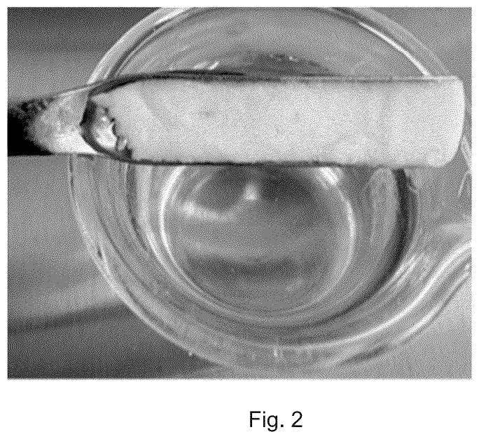

In the following, the admixability of antibiotics to the paste material according to the invention of example 1 was tested. The powdered antibiotics gentamicin hydrochloride, vancomycin hydrochloride, clindamycin hydrochloride and daptomycin were used. In each case, 5.0 g of paste from example 1 were kneaded with 0.5 g of the powdered antibiotic (FIG. 1). The haptic properties and adhesion to a steel surface (1.4404 steel) were tested. It was found that the kneaded antibiotics had only a small effect on the softness and kneadability compared to the pure paste of example 1. The kneadability was similar to the paste of example 2. All pastes adhered very well to 1.4404 steel (FIG. 2). The adhesion was similar to the paste of example 2.

TABLE 6
Results on the admixability of antibiotics

| | Composition | | Haptic | Adhesion on |
|---|---|---|---|---|
| Example | Paste | Antibiotic | Assessment | Steel |
| 13 | 5.0 g paste, | 0.5 g | Soft, but slightly | Adheres very |
| | example 1 | gentamicin | less than pure | well, similar to |
| | | sulfate | paste of example | the pure paste |
| | | | 1, easy to knead | of example 1 |
| 14 | 5.0 g paste, | 0.5 g | Soft, easy to | Adheres very |
| | example 1 | vancomycin | knead, similar to | well, similar to |
| | | hydrochloride | the paste of | the pure paste |
| | | | example 2 | of example 1 |
| 15 | 5.0 g paste, | 0.5 g | Soft, easy to | Adheres very |
| | example 1 | clindamycin | knead, similar to | well, similar to |
| | | hydrochloride | the paste of | the pure paste |
| | | | example 2 | of example 1 |
| 16 | 5.0 g paste, | 0.5 g | Soft, easy to | Adheres very |
| | example 1 | daptomycin | knead, similar to | well, similar to |
| | | | the paste of | the pure paste |
| | | | example 2, but | of example 1 |
| | | | slightly firmer | |

### Testing of Adhesion to Metal Surfaces in the Presence of Water

The pastes were tested on metal surfaces (1.4404 steel). For this purpose, approximately 2 g of samples of the examples were pressed onto a spatula surface. The pastes adhered to the metal surface (FIG. 2). The pastes adhering to the metal were stored in water at room temperature for 7 days. There was no dissolution. The pH value of the distilled water was in the range of pH 6.0-6.8.

What is claimed is:

1. A hemostatic agent in paste form, comprising
a) at least one particulate sugar alcohol;
b) at least one saturated triglyceride having a melting point greater than or equal to 40° C.; and,
c) at least one saturated triglyceride having a melting point of less than 0° C.

2. The hemostatic agent in paste form according to claim 1, comprising, in each case based on the total weight of the hemostatic agent,
a) 30 to 60 wt. % of at least one particulate sugar alcohol;
b) 20 to 35 wt. % of at least one saturated triglyceride having a melting point greater than or equal to 40° C.; and,
c) 20 to 35 wt. % of at least one saturated triglyceride having a melting point of less than 0° C.

3. The hemostatic agent in paste form according to claim 1, wherein it has a viability of greater than 70% in the MTT or XTT in vitro cytotoxicity test according to the ISO10993-5 standard at 100% v/v.

4. The hemostatic agent in paste form according to claim 1, wherein the particulate sugar alcohol is selected from alditols and ketols.

5. The hemostatic agent in paste form according to claim 1, wherein the saturated triglyceride having a melting point greater than or equal to 40° C. is selected from glycerol tristearate, glycerol tripalmitate, glycerol trilaurate and mixed triglycerides formed from stearate and/or palmitate and/or laurate and glycerol.

6. The hemostatic agent in paste form according to claim 1, wherein the saturated triglyceride having a melting point of less than 0° C. is selected from glycerol trioctanoate, glycerol tridecanoate or the glycerol fatty acid mixtures Miglyol 812 N, Miglyol 810 N and Miglyol 829 N.

7. The hemostatic agent in paste form according to claim 1, wherein the particulate sugar alcohol has a particle diameter of less than 100 μm, wherein the particle diameters are determined by sieve fractionation.

8. The hemostatic agent in paste form according to claim 1, wherein the hemostatic agent remains dimensionally stable in distilled water at room temperature over a period of 7 days.

9. An active agent release system in paste form comprising a hemostatic agent in paste form according to claim 1 and at least one pharmaceutical active agent, preferably a particulate pharmaceutical active agent, wherein the pharmaceutical active agent is preferably selected from anti-infectives, blood coagulation activators, fibrinolysis inhibitors, immunomodulators, steroid hormones and growth factors.

10. A hemostatic agent in paste form according to claim 1 for use as a drug.

11. A hemostatic agent in paste form according to claim 9 for use as a drug.

12. A hemostatic agent in paste form according to claim 1 for use in the treatment of damaged bone tissue.

13. A hemostatic agent in paste form according to claim 9 for use in the treatment of damaged bone tissue.

14. The hemostatic agent in paste form or the active agent release system in paste form according to claim 10 for use in the treatment of bone cavities by filling, preferably by filling infected and debrided medullary cavities and screw holes in bone tissue.

13. The hemostatic agent in paste form or the active agent release system in paste form according to claim 11 for use in the treatment of bone cavities by filling, preferably by filling infected and debrided medullary cavities and screw holes in bone tissue.

14. The hemostatic agent in paste form or the active agent release system in paste form according to claim 12 for use in the treatment of bone cavities by filling, preferably by filling infected and debrided medullary cavities and screw holes in bone tissue.

15. The hemostatic agent in paste form or the active agent release system in paste form according to claim 13 for use in the treatment of bone cavities by filling, preferably by filling infected and debrided medullary cavities and screw holes in bone tissue.

16. The hemostatic agent in paste form or the active agent release system in paste form according to claim 10, wherein the hemostatic agent in paste form or active agent release system in paste form is adhered to bone tissue surfaces and to surfaces of metal implants by applying pressure, wherein the hemostatic agent in paste form or active agent release system in paste form is preferably adhered to the surfaces of knee joint endoprostheses, hip joint endoprostheses, shoulder joint endoprostheses, intramedullary nails and osteosynthesis plates.

17. The hemostatic agent in paste form or the active agent release system in paste form according to claim 12, wherein the hemostatic agent in paste form or active agent release system in paste form is adhered to bone tissue surfaces and to surfaces of metal implants by applying pressure, wherein the hemostatic agent in paste form or active agent release system in paste form is preferably adhered to the surfaces of knee joint endoprostheses, hip joint endoprostheses, shoulder joint endoprostheses, intramedullary nails and osteosynthesis plates.

18. The hemostatic agent in paste form or the active agent release system in paste form according to claim 14, wherein the hemostatic agent in paste form or active agent release system in paste form is adhered to bone tissue surfaces and to surfaces of metal implants by applying pressure, wherein the hemostatic agent in paste form or active agent release system in paste form is preferably adhered to the surfaces of knee joint endoprostheses, hip joint endoprostheses, shoulder joint endoprostheses, intramedullary nails and osteosynthesis plates.

19. A method for producing a hemostatic agent in paste form according to claim 1, wherein at least one particulate sugar alcohol, at least one saturated triglyceride having a melting point greater than or equal to 40° C. and at least one saturated triglyceride having a melting point of less than 0° C. are placed in a mixing container and kneaded together.

20. The method according to claim 19, wherein at least one particulate pharmaceutical active agent is mixed with a hemostatic agent.
